(19) United States
(12) Patent Application Publication
(10) Pub. No.: US 2015/0157583 A1
(43) Pub. Date: Jun. 11, 2015

(54) Pharmaceutical Composition

(71) Applicant: Gary D. Bennett, Newport Beach, CA (US)

(72) Inventor: Gary D. Bennett, Newport Beach, CA (US)

(21) Appl. No.: 14/562,486

(22) Filed: Dec. 5, 2014

Related U.S. Application Data
(60) Provisional application No. 61/912,121, filed on Dec. 5, 2013.

(51) Int. Cl. A61K 31/14; A61K 45/06

(52) U.S. Cl. CPC A61K 31/14; A61K 45/06

(57) ABSTRACT

Pharmaceutical compositions comprising a plurality of ingredients that synergistically provide an antimicrobial, analgesic, or wound healing characteristic.

### CROSS REFERENCE TO RELATED APPLICATIONS

This is a U.S. Nonprovisional Application claiming priority from a U.S. Provisional Application having Ser. No. 61/912,121, filed on Dec. 5, 2013, which is hereby incorporated by reference.

### FIELD

Embodiments generally relate to a composition having pharmaceutical characteristics, and more particularly, to a composition that has antimicrobial, analgesic, or wound healing characteristics; and most particularly to a topical cream that has antimicrobial, analgesic, or wound healing characteristics.

### BACKGROUND

Bacterial infections are epidemic worldwide due to the development of bacterial strains that have developed resistant to available topical and systemic antibiotics. Currently available over the counter (OTC) or prescription topical antibiotics do not have a sufficiently broad spectrum to cover common infectious bacterial agents including Escherichia coli, Staphylococcus epidermis, Klebsiella pneumonia, Micrococcus luteus, or Salmonella typhimurium. This resistance is a result of the overutilization of the newest classes of antibiotics for illnesses not necessarily caused by a bacterial agent; use of these newer classes of medications for infections that could easily have been treated with an older antibiotic; and the exposure of humans to antibiotics that have contaminated the food chain either by industrial expose to animals for the purpose of increasing yield or by the wholesale dumping of these chemicals into the water supply.

Scientists are now at the crossroads, unable to create newer classes of antibiotics in light of human safety concerns. Many of the latest classes of antibiotics are associated with rare, but serious, side-effects such as ligamentous injuries to ciprofloxin. The race against the development of new classes of safe antibiotics against the development of bacterial resentence is lost due to current methods of development and human testing and delay in governmental approval of newer antibiotics. The end result will likely be a worldwide increase in devastating bacterial infections similar to conditions prior to the development of penicillin.

Accordingly, it would be an advance in the art of human or veterinary care to develop or manufacture an antimicrobial, analgesic, or wound healing medical composition.

### BRIEF DESCRIPTION OF THE DRAWINGS

The invention will be better understood from a reading of the following detailed description taken in conjunction with the drawings in which like reference designators are used to designate like elements, and in which:

FIG. 1 is a kill rate table for an exemplary medical composition including Ingredient A;

FIG. 2 is a kill rate table for an exemplary medical composition including Ingredients A and B;

FIG. 3 is a kill rate table for Bactroban® cream mupirocin calcium cream 2%;

FIG. 4 is a kill rate table for Neosporin® Bacitracin zinc-neomycin sulfate;

FIG. 5 is a kill rate table for an exemplary medical composition including Ingredients A and C;

FIG. 6 is a bar graph of the mean transepidermal water loss (TEWL) values over time when the composition, including Ingredients A and B, was used on an induced wound;

FIG. 7 is a bar graph of epidermal turnover time when the medical composition, including Ingredients A and B, was used on an induced wound;

FIG. 8 is a table of ingredients, with corresponding functionality, for a medical composition; and

FIG. 9 is a list of ingredients for a medical composition.

### DETAILED DESCRIPTION OF THE PREFERRED EMBODIMENTS

The invention is described in preferred embodiments in the following description with reference to the FIG.s, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Various embodiments provide medical compositions that have antimicrobial, analgesic, or wound healing characteristics in humans or animals. In certain embodiments, medical compositions include chemicals that function in a synergistic fashion. For example, one of more of the medical compositions can be used as a bacterice, viricide, fungicide, or parasiticide. To illustrate, various embodiments of the medical composition are each effective against Hepitidis, AIDS, Polio, Small Pox, Asprigeous, Lime Disease, Viketsia, Valley Fever, Parasitic disease, Abecan, vigitative Niger, e-coli, or acne. In some embodiments, the medical compositions promote wound healing. In some embodiments, the medical compositions are applied via a cream, location, foam, gel, rinse, capsule, spray, vapor, or other means, for example.

### Antimicrobial Inactive Ingredients: Ingredient A

In certain embodiments, a medical composition has antimicrobial characteristics, such as a bactericide or a bacteriostatic mechanisms of action. Here, the antimicrobial characteristics is due to inclusion of ingredients, such as a chloride, an ether, an ester, and a paraben (“Ingredient A”). For example, in one embodiment, a medical composition includes: Arachidyl alcohol, Benzalkonium chloride (e.g., about 0.1%), Behenyl alcohol, Cetearyl alcohol, Methylparaben, Phenoxyethanol, and Propylparaben.

In certain embodiments, the medical composition includes one or more of the following Ingredient A: Tea tree oil (e.g., between about 1 to 10 weight percent); Lemon Grass oil (e.g., between about 1 to 10 weight percent); Lemon oil (e.g., between about 1 to 10 weight percent); Cinnamon oil (e.g., between about 1 to 10 weight percent); Eucalyptus oil (e.g., between about 1 to 10 weight percent); Thyme whiteoil (e.g., between about 1 to 10 weight percent); Lavender oil (e.g., between about 1 to 10 weight percent); Grapefruit oil (e.g., between about 1 to 10 weight percent); Clove Bud oil (e.g., between about 1 to 10 weight percent); Sandalwood oil (e.g., between about 1 to 10 weight percent); Peppermint oil (e.g., between about 1 to 10 weight percent); Kunzea oil (e.g., between about 1 to 10 weight percent); and Sage oil (e.g., between about 1 to 10 weight percent).

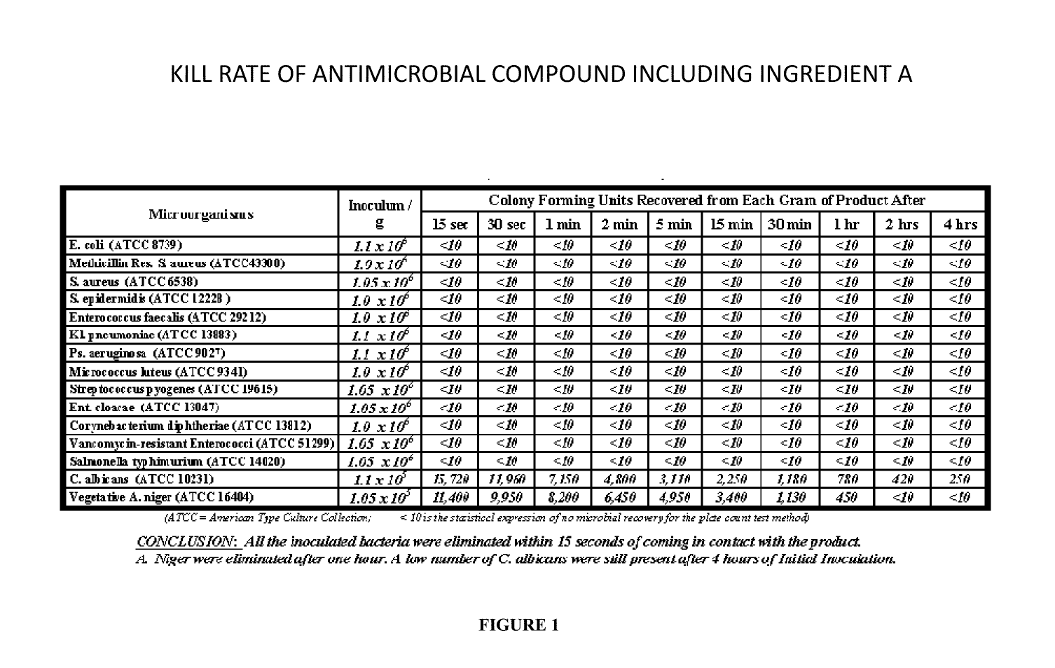

In certain embodiments, the medical composition including at least one Ingredient A works in synergy with other ingredients of the medical composition to exhibit antimicrobial characteristics. Referring to FIG. 1, a table illustrate the kill rate of a plurality of microorganisms as determined by an American Type Culture Collection (ATCC) testing standard for a medical composition including at least one Ingredient A. The kill rates evidences that the medical composition including at least one Ingredient A is effective in eliminating microbial inocula. All the inoculated bacteria were killed within one minute of coming in contact with the medical composition including at least one Ingredient A. For example, A albicans was eliminated within 5 minutes of coming in contact with medical composition including at least one Ingredient A.

### Antibiotic Active Ingredient: Ingredient B

In certain embodiments, the medical composition includes an antibiotic, such as neomycin (e.g., about 3.5 mg), bacitracin (e.g., about 500 units), polymyxin B sulfate (e.g., about 10,000 units), or any combination thereof (“Ingredient B”).

For example, three antibiotics: neomycin sulfate, bacitracin zinc, and polymyxcin B sulfate, is combined with the Ingredient A above and, optionally, other ingredients which are considered to be inactive by the United States Food and Drug Administration (FDA) that also exhibit bactericidal and bacterial static properties.

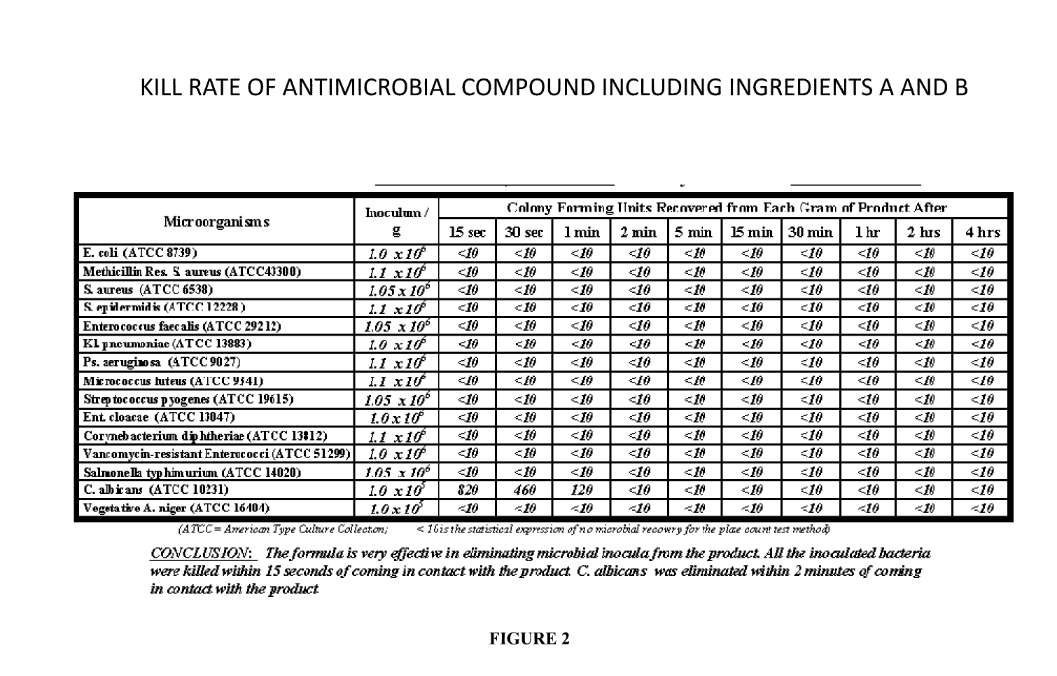

In certain embodiments, Ingredient A and Ingredient B within the medical composition work synergistically to exhibit antimicrobial characteristics. Referring to FIG. 2, a table illustrate the kill rate of a plurality of microorganisms as determined by an ATCC testing standard for a medical composition including at least one Ingredient A and at least one Ingredient B. The kill rates illustrate that the exemplary medical composition including at least one Ingredient A is effective in eliminating microbial inocula. Here, all the inoculated bacteria were killed within one minute of coming in contact with the medical composition including Ingredients A and B. A albicans was eliminated within 5 minutes of coming in contact with the medical composition including Ingredients A and B.

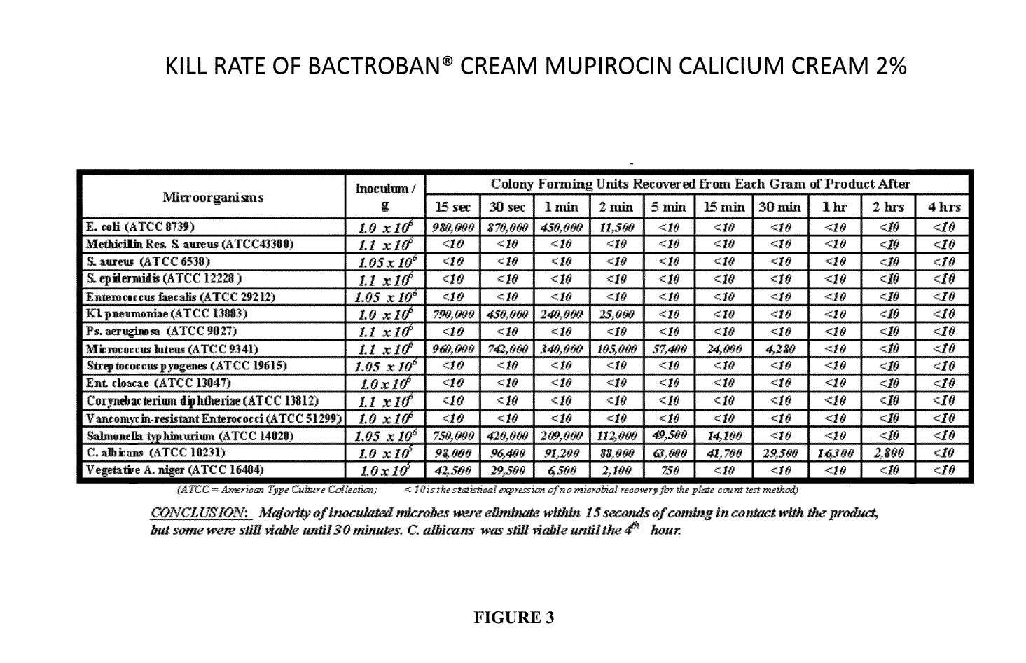

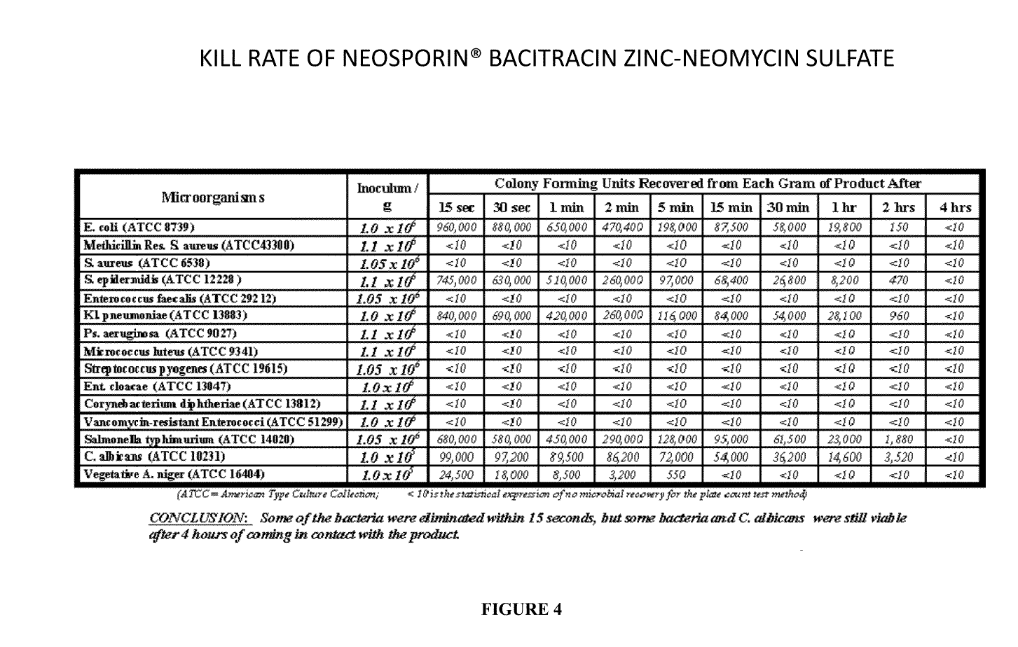

Referring to FIGS. 3 and 4, the kill rates of a plurality of microorganisms for each of Maximum strength Neosporin® antibacteria, which contains the same level of active antibiotics as those that produced the kill rates in FIG. 2, and Bactroban® are illustrated, respectively. A comparison of FIGS. 1-4 shows that the medical composition with at least one Ingredient A and the medical composition with at least one Ingredient A and at least one Ingredient B, each are more effective in killing the microbs than either Bactroban® or Neosporin®.

Therefore, in certain embodiments, a combination of at least one Ingredient A and at least one Ingredient B achieve a broader spectrum of bacterial killing than currently available OTC topical antibiotics without introducing a new classification of antibiotic medication while still complying with FDA OTC topical antimicrobial monographs.

### Antifungal Ingredient: Ingredient C

In certain embodiments, an exemplary medical composition has antifungal characteristics. The antimicrobial characteristics is due to inclusion of ingredients, such as Ingredient A described above. Alternatively, or in combination, the antifungal characteristics is caused, in part, by an ingredient such as Chlorhexidine Dihydrochloride (“Ingredient C”). To illustrate, the medical composition includes at least one Ingredient C in an amount ranging from about 0.1% to 5%, for example.

In certain embodiments, the medical composition includes one or more of the following Ingredient C: Tea tree oil (e.g., between about 1 to 10 weight percent); Lemon Grass oil (e.g., between about 1 to 10 weight percent); Lemon oil (e.g., between about 1 to 10 weight percent); Cinnamon oil (e.g., between about 1 to 10 weight percent); Eucalyptus oil (e.g., between about 1 to 10 weight percent); Thyme whiteoil (e.g., between about 1 to 10 weight percent); Lavender oil (e.g., between about 1 to 10 weight percent); Grapefruit oil (e.g., between about 1 to 10 weight percent); Clove Bud oil (e.g., between about 1 to 10 weight percent); Sandalwood oil (e.g., between about 1 to 10 weight percent); Peppermint oil (e.g., between about 1 to 10 weight percent); Kunzea oil (e.g., between about 1 to 10 weight percent); and Sage oil (e.g., between about 1 to 10 weight percent).

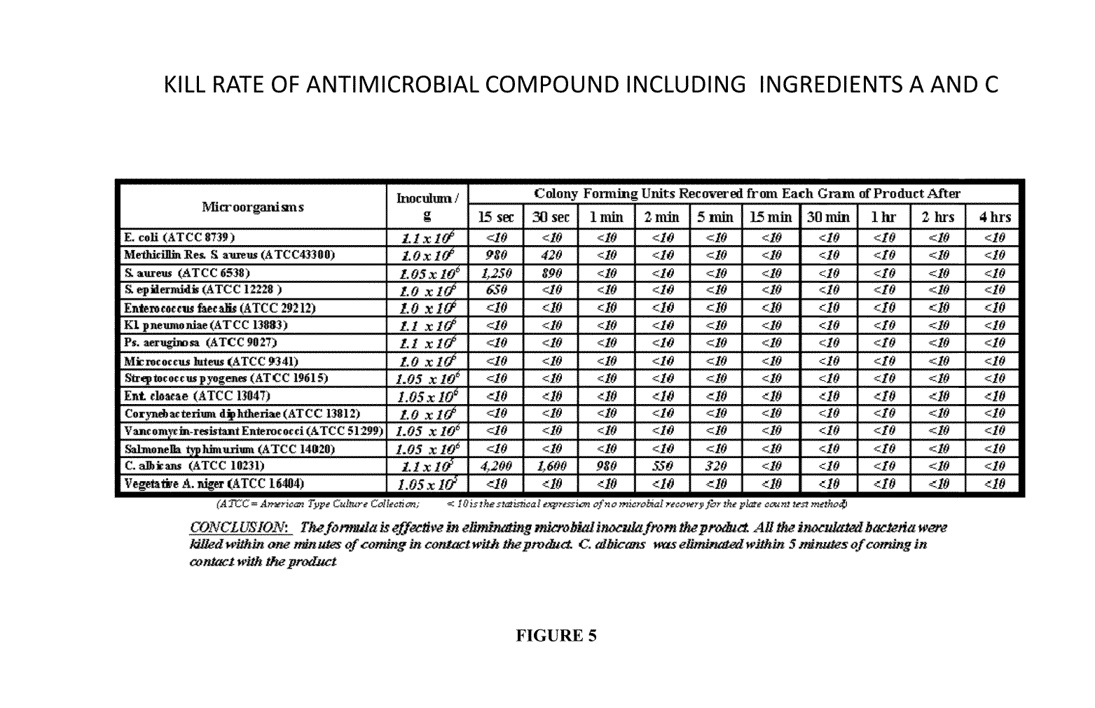

In certain embodiments, Ingredient A and Ingredient C work synergistically to exhibit antimicrobial characteristics. Referring to FIG. 5, a table illustrate the kill rate of a plurality of microorganisms as determined by an ATCC testing standard for a medical composition including at least one Ingredient A and at least one Ingredient C. The kill rates evidences that the medical composition including at least one Ingredient A and at least one Ingredient C is effective in eliminating microbial inocula. All the inoculated bacteria were killed within one minute of coming in contact with the medical composition including at least one Ingredient A and at least one Ingredient C. A albicans was eliminated within 5 minutes of coming in contact with medical composition including at least one Ingredient A and at least one Ingredient C.

Ingredient C alone, or in combination with Ingredient A, for example, can serve as an inexpensive and safe food surface or water decontaminate, especially in developing nations.

### Analgesic: Ingredient D

Most superficial wounds are associated with significant pain, particularly in the acute phase. The most common topical antibiotics, however, do not contain pain relieving ingredients.

In certain embodiments, the medical composition includes an analgesic (“Ingredient D”). To illustrate, an exemplary medical composition includes Lidocaine Hydrochloride (e.g., about 4%), benzocaine (e.g., about 20%), naturally occurring agents that have established scientific evidence demonstrating effectiveness against pain and/or inflammation, or a combination thereof. Examples of naturally occurring agents include: Aloe barbadensis leaf juice and Arnica montana flower extract.

### Cell Renewal Ingredient: Ingredient E

In certain embodiments, the medical composition includes ingredients that promote cell renewal or wound healing (“Ingredient E”) for minor cuts, wounds, abrasions, or burns. To illustrate, an exemplary medical composition includes one or more of the following ingredients:

Acrylates/Dimethicone copolymer

### Allantoin

Aloe barbadensis leaf juice
Arnica montana flower extract

### Ascorbic Acid

### 1,3 Beta-glucan

Butyrospermum parkii (shea butter)

### Dimethicone

Dipalmitoyl hydroxyproline
Helianthus annuus (Sunflower) seed oil
Hydrolyzed Mytilus edulis byssus

### Glycerin

Glycine soja (soybean) oil

### Panthenol

Persea gratissima (avocado) oil
Tocopherol (vitamin E)

The use of bovine collagen for wound healing is of concern due to the potential transmission of an infectious prion agent that results in Creutzfeldt-Jakob Disease or spongiform encephalopathy (BSE). This infectious agent is known to withstand all of the currently used sterilization techniques and is susceptible only to high pressure heat sterilization. Therefore, in certain embodiments, the Ingredient E included in the medical composition is a marine source of collagen, such as Hydrolyzed Mytilus Edulis Byssus.

The human epidermis represents a cell renewal system in which fully differentiated cells (corneocytes) are being continually shed from the skin surface. This system operates under steady state conditions, therefore, a loss of desquamated cells is balanced by new cell production in the germative cell layers.

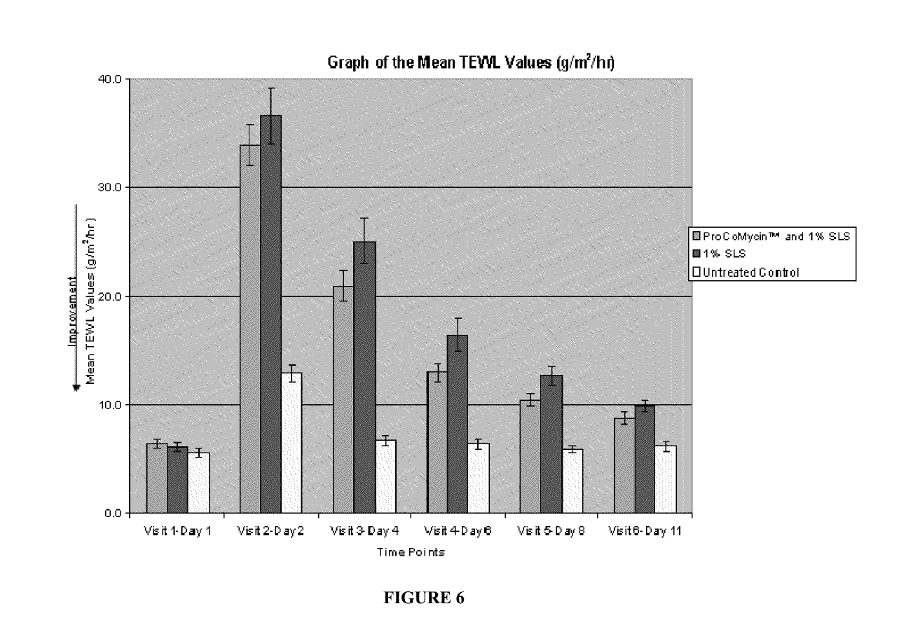

Referring to FIG. 6, a bar chart illustrates that a medical composition including Ingredient E, denoted as “ProComycin,” accelerated wound healing over control non-treated wounds by 38% in human subjects in which the transepidermal water loss (TEWL) was used as an indicator of barrier repair of the epidermis for induced skin lesions caused by sodium laurel sulfate.

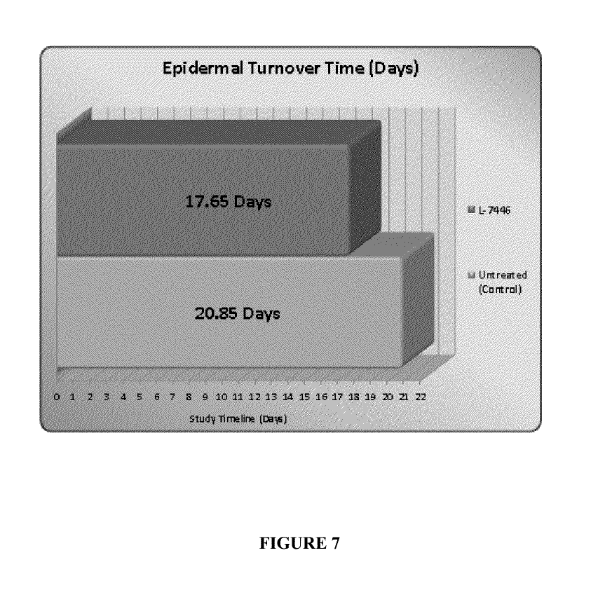

Transit time is the time required for a cell to move through a compartment and turnover time is the time required for a compartment to completely renew itself. In certain instances, transit time is equivalent to turnover time because the cells move in unison as a layer through the stratum corneum. Consequently, turnover time is a useful parameter for evaluating an efficacy of a medicament. Referring to FIG. 7, the epidermal turnover rate for subjects using the medical composition including Ingredient E demonstrated a 17% increased rate of epidermal turnover when compared with control subjects.

### Anticeptic Ingredient: Ingredient F

In certain embodiments, the medical composition includes an ingredient that is an anticeptic, such as a peroxide (“Ingredient F”). For example, in certain embodiments, a medical composition include at least one Ingredient A, at least one Ingredient F, or a combination thereof that, as topical agents, used as a treatment of acne vulgaris.

### Compositions Including Permutations of Ingredient

In certain embodiments, the medical compositions include any combination of the Ingredients A-F denoted above. To illustrate, an exemplary medical composition includes at least one of Ingredient A, at least one of Ingredient C, and at least one of Ingredient E. Other combinations of Ingredients A-E are also contemplated. Table 1 below lists a plurality of exemplary medical compositions including various permutations of the Ingredients A-F.

TABLE 1

| Composition Including | | | | | | |
|---|---|---|---|---|---|---|
| Ingredient: | A | B | C | D | E | F |
| A | | x | x | x | x | x |
| B | x | | x | x | x | x |
| C | x | x | | x | x | x |
| D | x | x | x | | x | x |
| E | x | x | x | x | | x |
| F | x | x | x | x | x | |

For example, the table cell denoted by column B and Row C represents a medical composition that includes at least one Ingredient B and at least one Ingredient C, and optionally, any other combination of the other Ingredients (such as an ingredient selected from the group including A, D, E, and F). For exemplary purposes, the weight percent of the Ingredients in Table 1 is denoted below by the following exemplary formula:

100=P(Ingredient A)+P(Ingredient B)+P(Ingredient C)+P(Ingredient D)+P(Ingredient E)+P(Ingredient F),+P(Water or other vehicle) where: Formula 1:
The P(Ingredient A) is any weight percentage value between 0% to 100% (e.g, 0, 5, 10, 15, 20, 25, 30, 35, 40, 45, 50, 55, 60, 65, 70, 75, 80, 85, 90, 95, 100, and values therebetween) that satisfies Formula 1;
P(Ingredient B) is any weight percentage value between 0% to 100% (e.g, 0, 5, 10, 15, 20, 25, 30, 35, 40, 45, 50, 55, 60, 65, 70, 75, 80, 85, 90, 95, 100, and values therebetween) that satisfies Formula 1;
P(Ingredient C) is any weight percentage value between 0% to 100% (e.g, 0, 5, 10, 15, 20, 25, 30, 35, 40, 45, 50, 55, 60, 65, 70, 75, 80, 85, 90, 95, 100, and values therebetween) that satisfies Formula 1;
P(Ingredient D) is any weight percentage value between 0% to 100% (e.g, 0, 5, 10, 15, 20, 25, 30, 35, 40, 45, 50, 55, 60, 65, 70, 75, 80, 85, 90, 95, 100, and values therebetween) that satisfies Formula 1;
P(Ingredient E) is any weight percentage value between 0% to 100% (e.g, 0, 5, 10, 15, 20, 25, 30, 35, 40, 45, 50, 55, 60, 65, 70, 75, 80, 85, 90, 95, 100, and values therebetween) that satisfies Formula 1;
P(Ingredient F) is any weight percentage value between 0% to 100% (e.g, 0, 5, 10, 15, 20, 25, 30, 35, 40, 45, 50, 55, 60, 65, 70, 75, 80, 85, 90, 95, 100, and values therebetween) that satisfies Formula 1; and
P(Water or other vehicle) is any weight percentage value between 0% to 100% (e.g, 0, 5, 10, 15, 20, 25, 30, 35, 40, 45, 50, 55, 60, 65, 70, 75, 80, 85, 90, 95, 100, and values therebetween) that satisfies Formula 1.
   Table 2 below shows several example compositions and the percentage weight of the corresponding ingredients:

TABLE 2

| | Weight Percentage of Ingredients | | | | | | | | | | |
|---|---|---|---|---|---|---|---|---|---|---|---|
| | For Exemplary Medical Compositions (“C1-C11”) | | | | | | | | | | |
| Ingredients | C1 | C2 | C3 | C4 | C5 | C6 | C7 | C8 | C9 | C10 | C11 |
| Water | 0 | 10 | 20 | 30 | 40 | 50 | 60 | 70 | 80 | 90 | 0 |
| A | 0 | 80 | 50.1 | 20.5 | 7 | 3 | 5 | 10 | 5 | 2.22 | 0 |
| B | 50 | 5 | 9 | 2 | 10.5 | 44 | 13.5 | 5 | 7 | 1 | 0 |
| C | 50 | 5 | 20.9 | 47.5 | 42.5 | 3 | 21.5 | 15 | 8 | 6.78 | 100 |

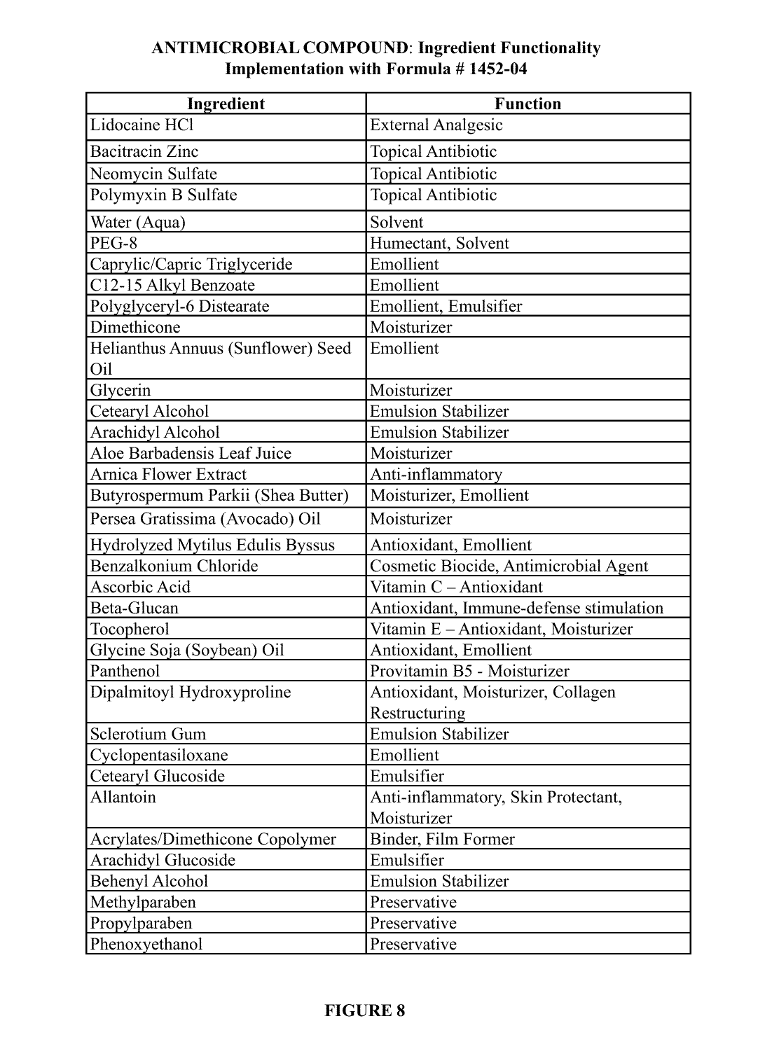

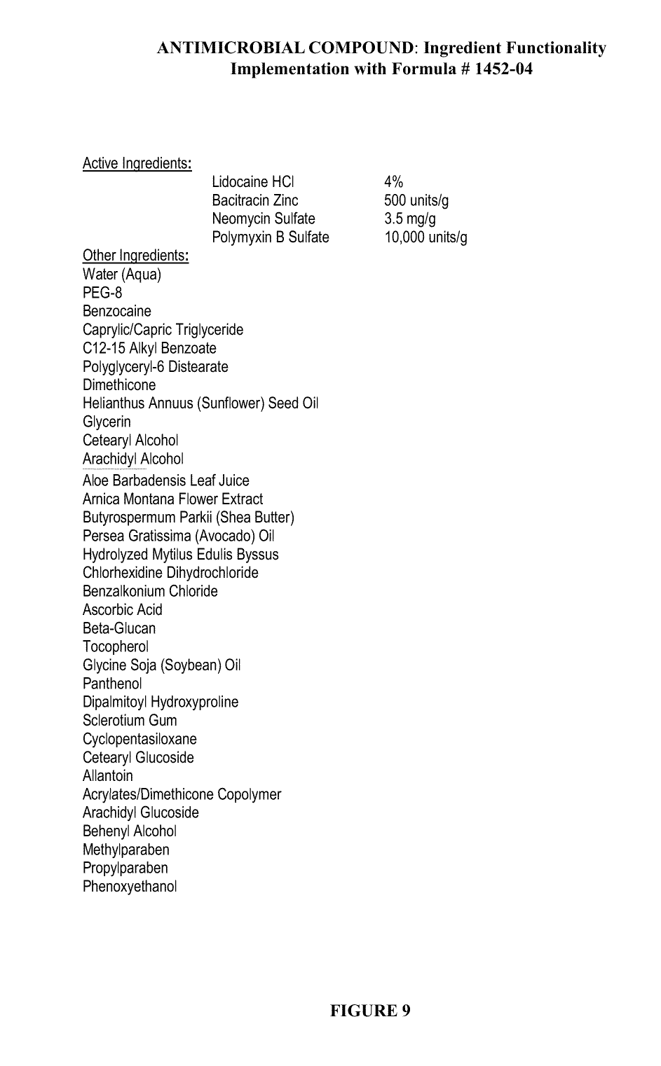

FIGS. 8-9 further illustrate exemplar medical compositions including a combination of the ingredients selected from the group including Ingredients A-F.

### Applications

As previously denoted, in certain embodiments, these medical compositions are used as a bacterice, viricide, fungicide, or parasiticide. They promote wound healing, acne treatment, act as an anticpentic hand sanitizer, toilet seat disinfectant, or water/food purifier, for example. Other uses for such compositions are also contemplated.

An exemplary medical composition is a water purifier that includes any or all of the following ingredients: at least one Ingredient C (about 0.1%); at least one Ingredient A; or any combination of Ingredients A, B, and C. To illustrate, a person traveling to a foreign land takes a vial of a medical composition including Ingredient C. Once there, the person adds a few drops of the medical composition including Ingredient C into a glass of water. Here, the traveler will be protected from various diseases and parasites that, perhaps the local residents have become immune to.

### Medium to Apply Medical Compositions

In certain embodiments, the medical compositions herein described are administered in any of a plurality of forms. To illustrate, an exemplary medical composition is administered as a cream, a lotion, a foam, a rinse, a tablet, a patch, a spray, a vapor, or other forms or means that are known in the art.

In certain embodiments, the medical composition including at least one Ingredient A and at least one Ingredient B is in the form of a Cream that is topically applied (e.g., about 1 ml to 5 ml) over a skin area (e.g., 2 cm×2 cm) thrice daily every 8 hours to promote wound healing.

### Producing the Composition

An exemplary method for producing a medical composition is described below:

PROCOMYCIN TOPICAL ANTIBIOTIC CREAM
Percentage Formula
Formula # 1452-04

| Trade Name | Wt. % |
|---|---|
| Deionized Water | 54.3800% |
| Activera 1-200C (Active Organics) | 0.0500% |
| Amigel (Alban Muller) | 0.8000% |
| Allantoin (DSM) | 0.2000% |
| Glycerine 99.7% USP (Univar) | 2.0000% |
| Methylparaben (Charkit) | 0.2000% |
| D-Panthenol 75W (BASF - Chemtec) | 0.1000% |
| Plurol Stearique WL 1009 (Gattefossé - Lipscomb) | 2.5000% |
| Propylparaben NF (Charkit) | 0.1000% |
| Finsolv TN (Innospec - Argan) | 3.0000% |
| Montanov 68 (Seppic) | 2.0000% |
| Montanov 202 (Seppic) | 2.0000% |
| Myritol 312 (Cognis) | 4.0000% |
| Avocado Oil (Desert Whale) | 1.0000% |
| Sunflower Oil (Desert Whale) | 2.0000% |
| Arnica Oil CLR (CLR - Deveraux) | 0.1000% |
| Shea Butter, Ultra Refined(BioChemica) | 1.0000% |
| Dimethisil DM-1,000 (Chemsil) | 2.0000% |
| KP-545 (Shin Etsu - Chemtec) | 1.0000% |
| Sepilift DPHP (Seppic) | 0.0500% |
| Carbowax PEG 400(Dow Company) | 9.0000% |
| Benzocaine USP (Spectrum) | 4.5000% |
| Bronidox 1160 (Cognis) | 0.7000% |
| Ascorbic Acid, Ultra Fine Powder(DSM) | 0.0100% |
| SymGlucan #151719 (Symrise) | 0.1000% |
| Mythuline (ISP) | 0.1000% |
| Stepanquat 50 NF (Stepan) | 0.0800% |
| Lidocaine HCl (Spectrum) | 4.9300% |
| Chlorhexidine Dihydrochloride (Spectrum) | 0.1000% |
| Polymixin B Sulfate USP (Santec Chemicals) | 0.1667% |
| Bacitracin Zinc USP(Santec Chemicals) | 1.2500% |
| Neomycin Sulfate USP (Santec Chemicals) | 0.5833% |
| | 100.0000% |

PHYSICIAN'S SCIENCE AND NATURE, INC.
PROCOMYCIN TOPICAL ANTIBIOTIC CREAM
Manufacturing Procedure
Formula # 1452-04

| | | |
|---|---|---|
| Item No. | Trade Name | Wt. % |

Phase A

| | | | |
|---|---|---|---|
| 1 | Deionized Water | 48.3800% | Main Processing Tank |
| 2 | Activera 1-200C | 0.0500% | |
| 3 | Amigel | 0.8000% | |
| 4 | Allantoin | 0.2000% | |
| 5 | Glycerine 99.7% USP | 2.0000% | |
| 6 | Methylparaben | 0.2000% | |
| 7 | D-Panthenol 75W | 0.1000% | |

Phase B

| | | | |
|---|---|---|---|
| 8 | Plural Stearique WL | 2.5000% | Auxiliary Tank |
| | 1009 | | |
| 9 | Propylparaben NF | 0.1000% | |
| 10 | Finsolv TN | 3.0000% | |
| 11 | Montanov 68 | 2.0000% | |
| 12 | Montanov 202 | 2.0000% | |
| 13 | Myritol 312 | 4.0000% | |
| 14 | Avocado Oil | 1.0000% | |
| 15 | Sunflower Oil | 2.0000% | |
| 16 | Arnica Oil CLR | 0.1000% | |
| 17 | Shea Butter, Ultra | 1.0000% | |
| | Refined | | |
| 18 | Dimethisil DM-1,000 | 2.0000% | |
| 19 | KP-545 | 1.0000% | |
| 20 | Sepilift DPHP | 0.0500% | |

Phase C

| | | | |
|---|---|---|---|
| 21 | Carbowax PEG 400 | 6.0000% | Auxiliary Tank |
| 22 | Benzocaine USP | 4.5000% | |

Phase D

| | | | |
|---|---|---|---|
| 23 | Bronidox 1160 | 0.7000% | Main Processing Tank |
| 24 | Ascorbic Acid, Ultra | 0.0100% | |
| | Fine Powder | | |
| 25 | SymGlucan #151719 | 0.1000% | |
| 26 | Mythuline | 0.1000% | |
| 27 | Stepanquat 50 NF | 0.0800% | |

Phase E

| | | | |
|---|---|---|---|
| 28 | Deionized Water | 5.0000% | Auxiliary Tank |
| 29 | Lidocaine HCl USP | 4.9300% | |

Phase F

| | | | |
|---|---|---|---|
| 30 | Deionized Water | 1.0000% | Auxiliary Tank |
| 31 | Chlorhexidine | 0.1000% | |
| | Dihydrochloride | | |

Phase G

| | | | |
|---|---|---|---|
| 32 | Carbowax PEG 400 | 3.0000% | Auxiliary Tank |
| 33 | Polymixin B Sulfate | 0.1667% | |
| | USP | | |
| 34 | Bacitracin Zinc USP | 1.2500% | |
| 35 | Neomycin Sulfate USP | 0.5833% | |

### Weighing Procedures

1. Wipe drum lids and/or bags to avoid dust and debris contamination.
2. Weigh/measure and check all ingredients into separate, clean, properly identified suitable size tared container(s).
3. Follow cGMP requirements.

### Compounding Procedures

1. Follow current good manufacturing practice (cGMP) requirements.
2. Clean and sanitize all equipment and tools that come in contact with the product prior to use.
3. Stage all materials to the main blending area. Check that all ingredients are approved for use and labeled properly.
4. Phase A: Into the main processing tank, add item #1 (DI Water). Start high speed mixing. Sprinkle in items #2 and #3. Heat to 85° C. Mix until completely hydrated. Add items #4-#7 in the given order, mixing well after each addition. Maintain temperature.
5. Phase B: In a separate tank, add items #8-#20. Heat to 85° C. Mix until uniform. Add Phase B to Phase A. Mix until uniform. Cool to 50° C.
6. Phase C: In a separate tank, add items #21 and #22. Heat to 70° C. Mix until all the solids are completely dissolved. Cool to 50° C. Add to the main tank. Mix until uniform. Cool to 40° C.
7. Phase D: At 40° C., add items #23-#27 in the given order, mixing well after each addition.
8. Phase E: In a separate container, add items #28 and #29. Mix until all the solids are completely dissolved. Add to the main tank. Mix until uniform.
9. Phase F: In a separate container, add items #30 and #31. Mix until all the solids are completely dissolved. Add to the main tank. Mix until uniform.
10. Phase G: In a separate container, add items #32-#35, mixing well after each addition. When uniform, add to the main tank.
11. QS batch with DI water, if necessary. Continue mixing and cooling to 35° C. Mix for 20 minutes or until uniform. Using a clean container, bring sample and the completed batch record to the laboratory for testing and approval.

### Filling Procedures

Follow cGMP requirements.

### Specifications for An Exemplary Medical Composition

Color: Off-white (to match standard)
Odor: Characteristic (to match standard)
Appearance: Opaque, viscous cream (to match standard)

### pH @ 25° C.: 4.80-5.50

Viscosity @ 25° C.: 15,000-20,000 cps (initial)
(RVT: Spindle 6 @ 10 rpm) 20,000-30,000 cps (after 24 hours)

### Specific Gravity @ 25° C.: 1.00-1.04

Total Aerobic Plate Count: Less than 100 cfu/g
Yeast & Mold: Less than 100 cfu/g

### P. Aeruginosa: Absent

### S. Aureus: Absent

Assay:

### Lidocaine HCl 4%±0.40%

### Bacitracin Zinc 1.25%±0.125%

### Neomycin Sulfate 0.5833%±0.05833%

### Polymyxin B Sulfate 0.1667%±0.01667%

### Supplementary Tests

IR: To match standard
% Solids @ 130° C.: 38.00-42.00
Another Exemplary Medical Composition is described as follows:

PHYSICIAN'S SCIENCE AND NATURE, INC.
PROCOMYCIN TOPICAL ANTIBIOTIC CREAM
Regulatory Data
Formula # 1452-04

| CAS # | Trade Name | INCI Name | Wt. % | Breakdown |
|---|---|---|---|---|
| 7732-18-5 | Deionized Water | Water (Aqua) | 54.3800% | 100% |
| 85507-69-3 | Activera 1-200C | Aloe Barbadensis | 0.0500% | 100% |
| | | Leaf Juice | | |
| 39464-87-4 | Amigel | Sclerotium Gum | 0.8000% | 100% |
| 97-59-6 | Allantoin | Allantoin | 0.2000% | 100% |
| 56-81-5 | Glycerine 99.7% | Glycerin | 2.0000% | 100% |
| | USP | | | |
| 99-76-3 | Methylparaben | Methylparaben | 0.2000% | 100% |
| 81-13-0 | D-Panthenol 75W | Panthenol | 0.1000% | 75% |
| 7732-18-5 | | Water (Aqua) | | 25% |
| 34424-97-0 | Plurol Stearique | Polyglyceryl-6 | 2.5000% | 100% |
| | WL 1009 | Distearate | | |
| 94-13-3 | Propylparaben NF | Propylparaben | 0.1000% | 100% |
| 68411-27-8 | Finsolv TN | C12-15 Alkyl | 3.0000% | 100% |
| | | Benzoate | | |
| 67762-27-0 | Montanov 68 | Cetearyl Alcohol | 2.0000% | 80% |
| N/A | | Cetearyl Glucoside | | 20% |
| 629-96-9 | Montanov 202 | Arachidyl Alcohol | 2.0000% | 55% |
| 661-19-8 | | Behenyl Alcohol | | 30% |
| N/A | | Arachidyl Glucoside | | 15% |
| 65381-09-1 | Myritol 312 | Caprylic/Capric | 4.0000% | 100% |
| | | Triglyceride | | |
| 8024-32-6 | Avocado Oil | Persea Gratissima | 1.0000% | 100% |
| | | (Avocado) Oil | | |
| 8001-21-6 | Sunflower Oil | Helianthus Annuus | 2.0000% | 100% |
| | | (Sunflower) Seed | | |
| | | Oil | | |
| 8001-22-7 | Arnica Oil CLR | Glycine Soja | 0.1000% | 96.9% |
| | | (Soybean) Oil | | |
| 68990-11-4 | | Arnica Montana | | 3% |
| | | Flower Extract | | |
| 10191-41-0 | | Tocopherol | | 0.1% |
| 68920-03-6 | Shea Butter, Ultra | Butyrospermum | 1.0000% | 100% |
| | Refined | Parkii (Shea) | | |
| | | Butter | | |
| 63148-62-9 | Dimethisil | Dimethicone | 2.0000% | 100% |
| | DM-1,000 | | | |
| 541-02-6 | KP-545 | Cyclopentasiloxane | 1.0000% | 70% |
| N/A | | Acrylates/Dimethicone | | 30% |
| | | Copolymer | | |
| 41672-81-5 | Sepilift DPHP | Dipalmitoyl | 0.0500% | 75-90% |
| | | Hydroxyproline | | |
| 5117-19-1 | Carbowax PEG 400 | PEG-8 | 9.0000% | 100% |
| 94-09-7 | Benzocaine USP | Benzocaine | 4.5000% | 100% |
| 122-99-6 | Bronidox 1160 | Phenoxyethanol | 0.7000% | 100% |
| 50-81-7 | Ascorbic Acid, | Ascorbic Acid | 0.0100% | 100% |
| | Ultra Fine Powder | | | |
| 7732-18-5 | SymGlucan #151719 | Water (Aqua) | 0.1000% | 94% |
| 56-81-5 | | Glycerin | | 5% |
| 53238-80-5 | | Beta-Glucan | | 1% |
| N/A | Mythuline | Hydrolyzed | 0.1000% | 5% |
| 7732-18-5 | | Mytilus Edulis | | 94.7% |
| | | Byssus | | |
| | | Water (Aqua) | | |
| 85409-22-9 | Stepanquat 50 NF | Benzalkonium | 0.0800% | 51% |
| | | Chloride | | |
| 7732-18-5 | | Water (Aqua) | | 44% |
| 6108-05-0 | Lidocaine HCl USP | Lidocaine HCl | 4.9300% | 100% |
| 3697-42-5 | Chlorhexidine | Chlorhexidine | 0.1000% | 100% |
| | Dihydrochloride | Dihydrochloride | | |
| 1405-20-5 | Polymixin B Sulfate | Polymyxin B | 0.1667% | 100% |
| | USP | Sulfate | | |
| 1405-89-6 | Bacitracin Zinc USP | Bacitracin Zinc | 1.2500% | 100% |
| 1405-10-3 | Neomycin Sulfate USP | Neomycin Sulfate | 0.5833% | 100% |

While the preferred embodiments of the present invention have been illustrated in detail, it should be apparent that modifications and adaptations to those embodiments may occur to one skilled in the art without departing from the scope of the present invention, which is defined by claims appended hereinbelow.

### APPENDIX

The Appendix of the Provisional Application is hereby incorporated by reference herein.

I claim:

1. A pharmaceutical composition, comprising:
Arachidyl alcohol;
Benzalkonium chloride;
Behenyl alcohol;
Cetearyl alcohol;
Methylparaben;
Phenoxyethanol; and
Propylparaben.

2. The composition of claim 1, further comprising: an antibiotic selected from the group consisting of neomycin sulfate, bacitracin zinc, polymyxcin B sulfate, and a combination thereof.

3. The composition of claim 1, further comprising Chlorhexidine Dihydrochloride.

4. The composition of claim 1, further comprising an ingredient selected from the group consisting of:
Acrylates/Dimethicone copolymer;
Allantoin;
Aloe barbadensis leaf juice;
Arnica montana flower extract;
Ascorbic Acid;
1,3 Beta-glucan;
Butyrospermum parkii;
Dimethicone;
Dipalmitoyl hydroxyproline;
Helianthus annuus seed oil;
Hydrolyzed Mytilus edulis byssus;
Glycerin;
Glycine soja oil;
Panthenol;
Persea gratissima; and
Tocopherol.
